(19) United States
(12) Patent Application Publication
(10) Pub. No.: US 2012/0095570 A1
(43) Pub. Date: Apr. 19, 2012

(54) Skin-Like Prosthesis Cover Having Substantially Permanently Attached and Releasably Attached Portions

(75) Inventor: Stuart H. Marquette, Encinitas, CA (US)

(73) Assignee: DAW Industries, Inc.

(21) Appl. No.: 12/907,538

(22) Filed: Oct. 19, 2010

(51) Int. Cl. A61F 2/74

(52) U.S. Cl. 623/27

(57) ABSTRACT

A prosthesis cover for protecting and cosmetically enhancing a prosthetic device. The prosthesis cover includes a flexible stocking portion overlapping with a flexible tubular portion. The flexible tubular portion is configured to be substantially permanently attached to the prosthetic device and to at least partially cover an outer surface of the prosthetic device. The flexible stocking portion is configured to be releasably attached to the flexible tubular portion, to at least partially overlap the flexible tubular portion, and to cover the remainder of the outer surface of the prosthetic device.

### BACKGROUND OF THE INVENTION

1. Field of the Invention

This invention relates to prosthesis coverings for an endoskeletal or exoskeletal prosthesis. More specifically, this invention is directed to an external, protective, and cosmetic flexible covering for a prosthesis, with a portion that is substantially permanently attached to the prosthesis and a portion that is releasably attached to the prosthesis.

2. Background of the Related Art

Advances in the science and art of creating prostheses—artificial replacements for missing body parts—has greatly increased the quality of life for myriad individuals, including young children with birth defects, soldiers returning from war, and those battling debilitating illnesses. For those who have lost a body part as a result of disease or injury, a prosthetic device provides a level of self-sufficiency and self-confidence that simply would not otherwise be possible.

Prosthetic limbs, and artificial legs in particular, have advanced from simple wooden pegs to complex mechanisms that closely mimic the function of the human leg, with appropriate articulation at the knee, ankle, and foot joints. These advanced prosthetics allow amputees to walk with a more natural gait, thus reducing the wear on the artificial leg and its joint mechanisms as well as reducing the pressure and wear on the amputee's body.

Typically, the prosthetic limb is attached to the amputee's body by a socket, which is custom-fitted to the remaining portion of the amputee's natural limb. The socket is also integrated with, or attached to, the internal structure of the prosthetic limb. The socket is usually made from a material such as polypropylene or laminated from a variety of fabrics impregnated with resins, and the internal structure of the prosthetic limb may be constructed from lightweight metal alloys or carbon fiber. The prosthetic limb may also include a covering.

Prosthesis coverings serve two purposes: they provide a natural look to the prosthesis by incorporating skin color and texture that match those of the amputee; and they protect the prosthesis and its mechanisms from the environment, thus increasing the useful life of the prosthesis. Coverings may either be formed as part of the prosthesis or as a removable element.

Soft covers made from materials such as polyurethane foam are commonly used as removable prosthesis coverings. However, use of these foam covers also requires use of a prosthetic stocking that goes over the foam cover to protect it. These stockings are prone to snags, they get dirty quickly, and they must be replaced frequently.

Alternatively, a permanent, non-removable finish may be applied to the prosthetic limb. Although this option provides for greater durability when compared to stockings, when the prosthesis—inevitably—needs to be adjusted or repaired, it must be refinished, which is an expensive and time-consuming process.

Another option is to use a skin-like prosthesis coverings having a straight cylindrical top and beads of silicone on the inside surface near the top. The silicone beads hold up the covering by friction alone, similar to the way that well-known thigh-high stockings are held up. These coverings are typically not trimmed to fit the curved upper edge of the prosthesis socket; doing so would cut away the resilient band and thus eliminate or reduce the frictional force required to keep the covering on the prosthesis. As a result, these coverings do not provide a uniform look along the entire length of the prosthesis.

Other prosthesis covers may include a flexible skin attached to the prosthesis in a semi-permanent manner using an adhesive and cut to fit the shape of the individual prosthesis. The flexible skin is typically in a sock-like shape with a length that is greater than the length of the prosthesis. The flexible skin is applied by stretching it over the prosthesis, and attaching it to the prosthesis with an adhesive. Any excess portion of the flexible skin can then be removed by cutting or grinding. This approach provides a uniform and cosmetically appealing look and has generally worked very well. But removing the flexible skin to repair or adjust the prosthesis or simply to replace the flexible skin due to normal wear or accidental damage has proven to be problematic; the adhesive is not easily removed, thus marring the outer surface of the prosthesis.

Accordingly, there is a need for methods and systems for easily applying, removing, and re-applying a new flexible skin to a prosthetic device. The skin-like prosthesis cover described in this disclosure, having a substantially permanently attached portion and a releasably attached portion, meets this need.

### SUMMARY OF THE INVENTION

To achieve these and other advantages and in accordance with the purpose of the invention, as embodied herein, the invention includes a prosthesis cover having a flexible tubular portion configured to be substantially permanently attached to a prosthetic device and to at least partially cover an outer surface of the prosthetic device. The prosthesis cover also includes a flexible stocking portion configured to be releasably attached to the flexible tubular portion, to at least partially overlap the flexible tubular portion, and to cover the remainder of the outer surface of the prosthetic device.

A method of covering a prosthetic device is also disclosed. The method includes the steps of stretching a flexible sleeve around a prosthetic device such that a portion of the flexible sleeve surrounds the prosthetic device for at least a portion of the length of the prosthetic device and such that the flexible sleeve extends above a socket of the prosthetic device; substantially permanently attaching the flexible sleeve to an outer surface of the prosthetic device; trimming the portion of the flexible sleeve that extends above the socket to match a contour of the socket; stretching a flexible stocking of the prosthesis cover to surround the remainder of the outer surface of the prosthetic device; and releasably attaching the flexible stocking of the prosthesis cover to the flexible sleeve of the prosthesis cover in an overlapping manner.

A prosthetic device is also disclosed. The prosthetic device includes an a outer surface, a flexible tubular portion substantially permanently attached to the outer surface, and a flexible stocking portion releasably attached to the flexible tubular portion. The flexible tubular portion at least partially covers the outer surface of the prosthetic device, and the flexible stocking portion overlaps with at least a portion of the flexible tubular portion to cover the remainder of the outer surface of the prosthetic device.

### BRIEF DESCRIPTION OF THE DRAWINGS

So that those skilled in the art will readily understand how the skin-like prosthesis cover having a substantially permanently attached portion and a releasably attached portion functions, preferred embodiments of the prosthesis cover and methods of applying the cover are described in detail below with reference to the following figures:

FIG. 1 is a perspective view of a below-knee prosthetic leg without a protective covering;

FIG. 2 is a perspective view of the prosthetic leg of FIG. 1 with a two-part prosthesis cover including a flexible tubular portion substantially permanently attached to an upper portion of the prosthetic leg and a flexible stocking portion releasably attached to a lower portion of the leg;

FIG. 3 is perspective view of the prosthetic leg shown in FIG. 1 with a flexible tubular portion of a prosthesis cover substantially permanently attached to an upper portion of the leg and surrounding a socket of the leg, with excess material from the flexible tubular portion extending upward above the socket;

FIG. 4 is a perspective view of the prosthetic leg shown in FIG. 3, with the flexible tubular portion of the prosthesis cover trimmed to fit the contour of the socket of the leg;

FIG. 5 is a perspective view of the prosthetic leg of FIG. 4, illustrating the application of a flexible stocking portion of the prosthesis cover to the leg in an overlapping manner with respect to the flexible tubular portion;

FIG. 6 is a cross-sectional view taken along the line 6-6 shown in FIG. 2, illustrating a first adhesive layer between the flexible stocking portion of the prosthesis cover and the flexible tubular portion of the prosthesis cover, and a second adhesive layer between the flexible tubular portion and the leg;

FIG. 7 is an alternative embodiment of the flexible stocking portion of the prosthesis cover having an elastic band near the top of the stocking portion to grip and hold the stocking onto the leg; and

FIG. 8 is a perspective view of an above-knee prosthetic leg with a prosthesis cover having a flexible stocking portion applied to the leg and releasably adhered in an overlapping fashion to a flexible tubular portion of the prosthesis cover that is substantially permanently adhered to a top portion of the leg.

### DETAILED DESCRIPTION OF PREFERRED EMBODIMENTS

Both the functionality and physical appearance of a prosthetic limb are important to an amputee. The skin-like prosthesis cover described in this disclosure enhances the durability as well as the appearance of prosthetic limbs. The cover protects the prosthetic limb from wear and tear while at the same time creating a natural, smooth skin look. Advantageously, the prosthesis cover has both a substantially permanently attached portion near the socket of the prosthetic limb, providing a good fit between the amputee's residual limb and the prosthesis; and a releasably attached portion, which allows the skin-like cover to easily be removed without marring the surface of the prosthetic limb. When the releasably attached portion needs to be replaced due to normal wear and tear or accidental damage, or when the prosthetic limb needs to be repaired or adjusted, the releasably attached portion can easily be replaced without the need to replace the substantially permanently attached portion, thus avoiding the time-consuming gluing and trimming operations needed to fit the skin-like cover to the curved edge of the socket of the prosthetic limb.

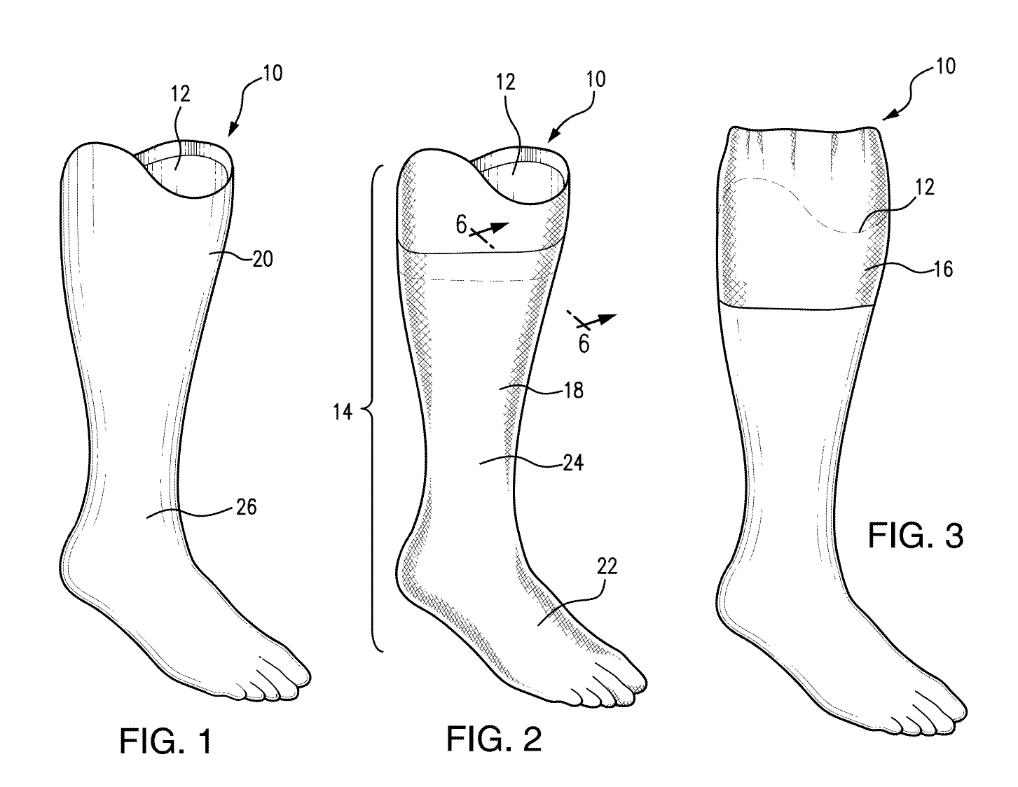

FIG. 1 illustrates a perspective view of a prosthetic device 10. Although the prosthetic device shown in FIGS. 1-7 of the drawings is a below-knee artificial leg, the prosthesis cover described in this disclosure may be used with any suitable prosthetic device, including without limitation a prosthetic leg, arm, hand, foot, finger, and toe; and with prosthetic devices of any shape, size, and construction. For example, the prosthesis cover may be configured for use with an above-knee prosthetic leg as shown in FIG. 8.

Prosthetic device 10 is typically molded from a light-weight, plastic material such as polypropylene or laminated from a variety of fabrics and resins. Device 10 includes a socket 12, which is custom-molded to fit the residual limb of an amputee. Socket 12 is contoured to provide a secure fit with the residual limb of the amputee, thus allowing the amputee to comfortably use prosthetic device 10 for extended periods.

FIG. 2 shows a prosthesis cover 14 of the present invention in place over prosthetic device 10. The cover not only protects prosthetic device 10 from the impacts, humidity, dust, dirt, and moisture it will encounter in everyday use, but also provides a realistic-looking approximation of human skin. In one exemplary embodiment, shown in FIG. 2, prosthesis cover 14 comprises two discrete parts: a flexible tubular portion 16 and a flexible stocking portion 18. Both portions of prosthesis cover 14 may be made of a flexible polyvinyl resin material marketed as DAWSKIN and available from Daw Industries of San Diego, Calif., USA. However, other suitable materials may also be used.

Prosthesis cover 14 may be manufactured by dipping, spraying, atomizing, cascading, or rolling a mold of a limb or other body part to be covered into suitable material, such as polyvinyl resin. The material is then cured on the mold to form the individual portions of prosthesis cover 14, which can then be removed from the mold and applied to prosthetic device 10.

Flexible tubular portion 16 of prosthesis cover 14 may be in the form of a sleeve that can be stretched and fitted over an upper portion 20 of prosthetic device 10. The material of tubular portion 16 may be substantially permanently attached to upper portion 20 with an adhesive. As used in this disclosure, the term “substantially permanently attached” or “substantially permanently adhered” means “not intended to be removed by the user in the normal course of use, but still professionally removable if needed to perform more involved repairs or other procedures to the prosthetic device.”

Flexible stocking portion 18 of prosthesis cover 14 may be in the form of a stocking or sock, that is, a tubular structure with one closed end. When used with a below-knee prosthetic device, as shown in FIG. 2, flexible stocking portion 18 may include both a foot portion 22 for accommodating the foot area of prosthetic device 10, and a leg portion 24 for accommodating a lower portion 26 of prosthetic device 10. Flexible stocking portion 18 may be stretched over prosthetic device 10 until it overlaps with flexible tubular portion 16; flexible stocking portion is then secured to flexible tubular portion 16 rather than being secured directly to prosthetic device 10.

In one exemplary embodiment, flexible stocking portion 18 is releasably secured to a lower part of flexible tubular portion 16 using a non-permanent adhesive or using one or more bands made of silicone or other elastic material, formed on or in flexible stocking portion 18, that will grip the lower part of tubular portion 16. The silicone bands may be formed on flexible stocking portion 18, similar to prior-art stocking-retention means.

FIGS. 3 through 5 illustrate the application of the two-part prosthesis cover 14 to prosthetic device 10. First, as shown in FIG. 3, flexible tubular portion 16 is stretched to fit around upper portion 20 of prosthetic device 10. An adhesive may be applied to upper portion 20 prior to the application of flexible tubular portion 16 to substantially permanently secure flexible tubular portion 16 to the prosthetic device. As shown in FIG. 3, flexible tubular portion 16 is stretched around prosthetic device 10 such that the flexible tubular portion extends above the brim of socket 12, which is indicated by a dotted line in the figure.

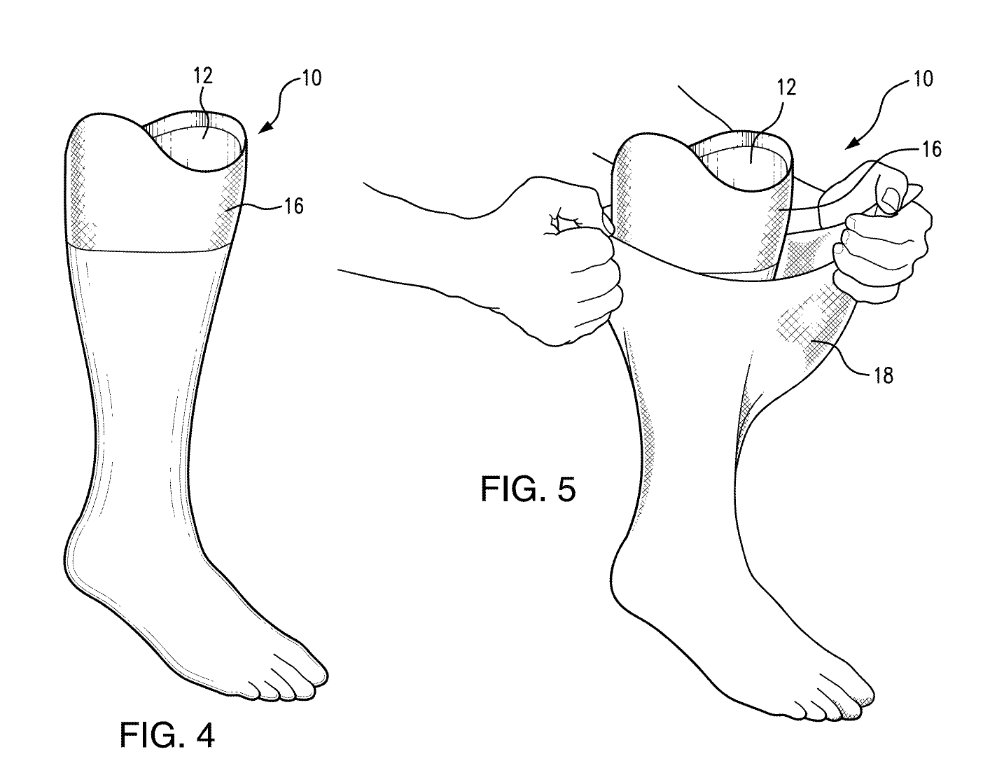

FIG. 4 shows a perspective view of prosthetic device 10 after flexible tubular portion 16 of prosthesis cover 14 has been trimmed to fit the contour of socket 12 of the prosthetic device. Tubular portion 16 may be cut to size by any suitable method. In one exemplary embodiment, an adhesive is applied directly to the outer surface of upper portion 20 of prosthetic device 10, and tubular portion 16 of the prosthesis cover is stretched over upper portion 20 of prosthetic device 10. Once the adhesive has set, tubular portion 16 can then be cut to fit. For example, the tubular portion 16 may be cut to fit around the contours of socket 12 of prosthetic device 10. The adhesive used may be any suitable adhesive. In one exemplary embodiment, the adhesive includes a cyanoacrylate composition that substantially permanently adheres the tubular portion 16 to upper portion 20 of prosthetic device 10.

FIG. 5 illustrates the application of flexible stocking portion 18 to prosthesis device 10 after flexible tubular portion 16 has been substantially permanently attached to upper portion 20 of the device. As shown, stocking portion 18 is stretched over lower portion 26 of the prosthetic device until it meets tubular portion 16 of prosthetic device 10. Stocking portion 18 is then secured to prosthetic device 10 by releasably attaching an inner surface of stocking portion 18 to an outer surface of tubular portion 16 such that there is some overlap between the two portions, as indicated by the dotted line shown in FIG. 2.

A releasable adhesive may be used to attach stocking portion 18 to tubular portion 16. For example, the adhesive may be a cyanoacrylate composition that is diluted to enhance the releasability of the adhesive. In one exemplary embodiment, the adhesive comprises a cyanoacrylate glue diluted with acetone to form a solution that is 70% acetone and 30% cyanoacrylate glue. The same cyanoacrylate glue, without dilution, may be used to substantially permanently attach tubular portion 16 directly to prosthetic device 10.

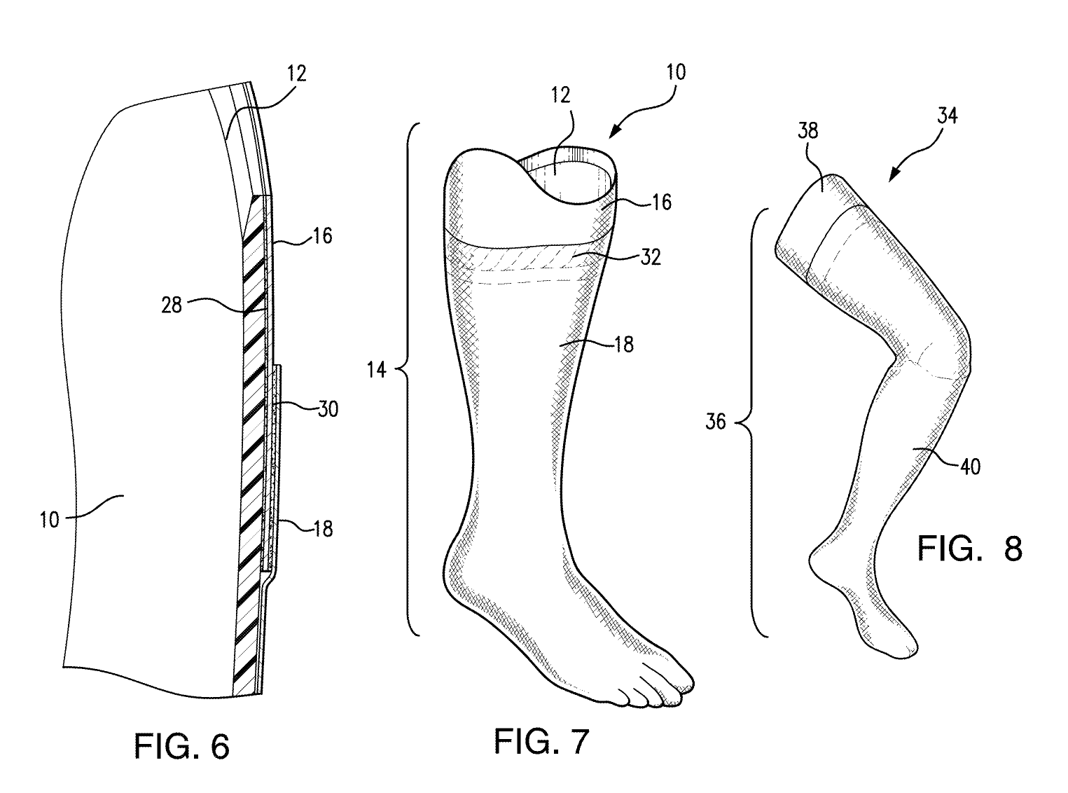

FIG. 6 is a cross-sectional view taken along the line 6-6 shown in FIG. 2, illustrating a first adhesive layer 28 between an inner surface of flexible tubular portion 16 and an outer surface of prosthetic device 10, and a second adhesive layer 30 between an inner surface of flexible stocking portion 18 and an outer surface of flexible tubular portion 16. In one exemplary embodiment, first adhesive layer 28 comprises a substantially permanent adhesive, and second adhesive layer 30 comprises a releasable adhesive, that is, an adhesive that allows flexible stocking portion 18 to be easily removed from flexible tubular portion 16.

FIG. 7 illustrates an additional embodiment of a two-part, below-knee prosthesis cover. In this embodiment, flexible stocking portion 18 of the prosthesis cover has a bead or band 32, made of silicone or another elastic material formed on the inner surface or within flexible stocking portion 18 to grip and releasably hold stocking portion 18 onto flexible tubular portion 16, thus securing the prosthesis cover on prosthetic device 10.

FIG. 8 is a perspective view of an above-knee prosthetic leg 34 covered with a two-part prosthesis cover 36 that includes a flexible tubular portion 38 substantially permanently adhered to an upper portion of the leg 34 and a flexible stocking portion 40 releasably adhered to the flexible tubular portion 38. The two-part prosthesis cover 36 for the above-knee prosthetic leg functions in the same way as the prosthesis cover 14 described above with respect to the below-knee prosthetic device; prosthesis cover 36 is simply sized to correctly fit the above-knee prosthetic device. In a similar manner, the prosthesis cover described in this disclosure can be a sized to fit any number of prosthetic devices. The two-part prosthesis cover described in this application allows an amputee to more quickly and easily change the prosthesis cover when desired. Removal of the cover may be necessary when the prosthetic limb needs to be repaired or altered, when the prosthesis cover needs replacement due to normal wear or accidental damage, or when the amputee simply wishes to change the color or appearance of the cover. Because the prosthesis cover is divided into two portions, there is no need for time-consuming and labor-intensive sizing and cutting when the cover is removed; the tubular portion remains in place, and the stocking portion is replaced.

It will be apparent to those skilled in the art that various modifications and variations can be made to the described prosthesis cover without departing from the scope of the invention as outlined in the appended claims and their equivalents.

The invention claimed is:

1. A prosthesis cover, comprising:
a flexible tubular portion configured to be substantially permanently attached to a prosthetic device and to at least partially cover an outer surface of the prosthetic device;
a flexible stocking portion configured to be releasably attached to the flexible tubular portion, to at least partially overlap the flexible tubular portion, and to cover the remainder of the outer surface of the prosthetic device.

2. The prosthesis cover of claim 1, further comprising a first adhesive for substantially permanently attaching the flexible tubular portion to the outer surface of the prosthetic device.

3. The prosthesis cover of claim 2, wherein the first adhesive comprises a cyanoacrylate composition.

4. The prosthesis cover of claim 1, further comprising a second adhesive for releasably attaching the flexible stocking portion to the flexible tubular portion.

5. The prosthesis cover of claim 4, wherein the second adhesive comprises a diluted cyanoacrylate composition.

6. The prosthesis cover of claim 5, wherein the cyanoacrylate composition is diluted with acetone.

7. The prosthesis cover of claim 6, wherein the second adhesive consists of 70% acetone and 30% cyanoacrylate composition.

8. The prosthesis cover of claim 1, wherein the flexible tubular portion is made from a material comprising a polyvinyl resin.

9. The prosthesis cover of claim 1, wherein the flexible stocking portion is made from a material comprising a polyvinyl resin.

10. The prosthesis cover of claim 1, wherein the prosthetic device is a below-knee prosthetic leg.

11. The prosthesis cover of claim 1, wherein the prosthetic device is an above-knee prosthetic leg.

12. The prosthesis cover of claim 1, wherein the flexible stocking portion further comprises an elastic band configured to releasably and securely attach the flexible stocking portion to the flexible tubular portion.

13. A method of covering a prosthetic device, the method comprising the steps of:
stretching a flexible sleeve around a prosthetic device such that a portion of the flexible sleeve surrounds the prosthetic device for at least a portion of the length of the prosthetic device and such that the flexible sleeve extends above a socket of the prosthetic device;
substantially permanently attaching the flexible sleeve to an outer surface of the prosthetic device;
trimming the portion of the flexible sleeve that extends above the socket to match a contour of the socket;
stretching a flexible stocking of the prosthesis cover to surround the remainder of the outer surface of the prosthetic device;
releasably attaching the flexible stocking of the prosthesis cover to the flexible sleeve of the prosthesis cover in an overlapping manner.

14. The method of claim 13, wherein the step of attaching the flexible sleeve to the outer surface of the prosthetic device comprises using an adhesive to substantially permanently adhere the flexible sleeve to the outer surface.

15. The method of claim 13, wherein the step of attaching the flexible stocking to flexible sleeve comprises using an adhesive to releasably adhere the flexible stocking to the flexible sleeve.

16. The method of claim 15, wherein the adhesive comprises a cyanoacrylate composition diluted with acetone.

17. The method of claim 15, wherein the adhesive consists of 70% acetone and 30% cyanoacrylate composition.

18. A prosthetic device, comprising:
an outer surface;
a flexible tubular portion substantially permanently attached to the outer surface of the prosthetic device, wherein the flexible tubular portion at least partially covers the prosthetic device; and
a flexible stocking portion releasably attached to the flexible tubular portion, wherein the flexible stocking portion overlaps with at least a portion of the flexible tubular portion to cover the remainder of the outer surface of the prosthetic device.

19. The prosthetic device of claim 1, wherein the prosthetic device is a below-knee prosthetic leg.

20. The prosthesis device of claim 1, wherein the prosthetic device is an above-knee prosthetic leg.
